(19) United States
(12) Patent Application Publication
(10) Pub. No.: US 2011/0251463 A1
(43) Pub. Date: Oct. 13, 2011

(54) BARIATRIC FOAM PORT

(75) Inventor: Gennady Kleyman, Brooklyn, NY (US)

(73) Assignee: Tyco Healthcare Group LP

(21) Appl. No.: 13/031,346

(22) Filed: Feb. 21, 2011

Related U.S. Application Data
(60) Provisional application No. 61/323,013, filed on Apr. 12, 2010.

(51) Int. Cl. A61B 1/32

(52) U.S. Cl. 600/205

(57) ABSTRACT

A surgical apparatus for positioning within a tissue tract accessing an underlying body cavity is adapted to tissues having different thicknesses. The surgical apparatus is configured to have different lengths. In one embodiment, the surgical includes a seal anchor member having two ends, one of which can be configured to fold and result in a plurality of folded states. Each folded state corresponds to a different length of the seal anchor member. The seal anchor member includes a slot to facilitate transition within the plurality of folded states. The seal anchor member further includes an aperture and a pin configured to further facilitate transition within the plurality of folded states.

### CROSS-REFERENCE TO RELATED APPLICATIONS

This application claims priority to, and the benefit of, U.S. Provisional Patent Application Ser. No. 61/323,013, filed on Apr. 12, 2010, the entire contents of which is hereby incorporated by reference.

### BACKGROUND

1. Technical Field

The present disclosure relates generally to surgical apparatuses for use in minimally invasive surgical procedures, such as endoscopic and/or laparoscopic procedures, and more particularly, relates to a surgical apparatus that allows multiple surgical instruments to be inserted through a single incision.

2. Description of Related Art

Today, many surgical procedures are performed through small incisions in the skin, as compared to the larger incisions typically required in traditional procedures, in an effort to reduce both trauma to the patient and recovery time. Generally, such procedures are referred to as “endoscopic”, unless performed on the patient's abdomen, in which case the procedure is referred to as “laparoscopic.” Throughout the present disclosure, the term “minimally invasive” should be understood to encompass both endoscopic and laparoscopic procedures.

During a typical minimally invasive procedure, surgical objects, such as surgical access devices (e.g., trocar and cannula assemblies) or endoscopes, are inserted into the patient's body through the incision in tissue. In general, prior to the introduction of the surgical object into the patient's body, insufflation gas is used to enlarge the area surrounding the target surgical site to create a larger, more accessible work area. Accordingly, the maintenance of a substantially fluid-tight seal is desirable so as to inhibit the escape of the insufflation gas and the deflation or collapse of the enlarged surgical site.

Different patients have different tissue thicknesses. Generally, access devices of different lengths are supplied in order to meet the various demands of patients based on their various tissue thicknesses. Such prior access devices as have been heretofore provided have had numerous disadvantages both from the standpoint of design as well as from the standpoint of availability of use.

From the design perspective, no one single prior access device is universally suitable for tissues having different thicknesses. In the prior art, each access device is designed in contemplation of a tissue having a particular thickness. Thus, access devices of different lengths have to be designed and supplied in order to accommodate patients with different needs based on their tissue thicknesses.

In the use of prior access devices, patient's tissue thickness needs to be assessed before performing a minimally invasive procedure. After assessing the tissue thickness and before performing the procedure, an access device having a length suitable for the patient's tissue thickness is selected. An error made in the assessment may lead to consequences adversely impacting the procedure. For instance, if the assessment underestimates the patent's tissue thickness, then an access device having a length less than the patient's tissue thickness may be selected. As a result, the selected access device is inadequate for the procedure, thus delaying the performance of the procedure.

Thus, to avoid the need of designing and supplying access devices of different lengths, to preclude the need of assessing tissue thickness, and to avoid unnecessary problems caused by the assessment, it is desirable to have a single access device that can be configured to different lengths, such that the single access device can be suitable for tissues having different thicknesses.

### SUMMARY

Disclosed herein is a surgical apparatus for positioning within a tissue tract accessing an underlying body cavity. The surgical apparatus comprises a seal anchor member. The seal anchor member has a longitudinal axis, a length, a first end and a second end. The first end is configured to fold along the longitudinal axis, resulting in a plurality of states. Each state corresponds to a different length of the seal anchor member.

In one embodiment, the plurality of states comprises a plurality of folded states. In each folded state, the first end has an outer surface and an inner surface. Each folded state is maintained by connecting the outer surface and the inner surface of the first end together. The plurality of folded states includes a maximum folded state and a minimum folded state. In the maximum folded state, the length of the seal anchor member is minimized. Likewise, in the minimum folded state, the length of the seal anchor member is maximized.

In another embodiment, the plurality of states further comprises an unfolded state in which the first end of the seal anchor member is not folded. Similar to the embodiment described above, the length of the seal anchor is minimized in the maximum folded state. Unlike the embodiment described above, the length of the seal anchor member is maximized in the unfolded state.

In a certain embodiment, the first end of the seal anchor member defines a slot to facilitate transition among the plurality of folded states. Further, the first end defines an aperture through which a pin is used to further facilitate transition between the maximum and minimum folded states. The pin further connects the outer surface and the inner surface together to maintain a folded state.

In another embodiment, a surgeon manually adjusts the length of the outer surface and the length of the inner surface of the first end in order to select a desired folded state. After making the selection, the surgeon uses a suture to hold the inner surface and the outer surface of the first end together for purposes of maintaining the selected folded state.

In a certain embodiment, the first end defines a substantially large radial diameter thereby increasing the range of motion of the surgical instruments inserted through the seal anchor member. The second end defines a substantially small radial diameter thereby providing easy insertion and removal of the seal anchor member through the tissues.

In a further embodiment, the length of the seal anchor member is substantially large for accommodating thick tissues in bariatric related procedures.

In another embodiment, the seal anchor member defines a coring configuration such that there is a large free open space within the seal anchor member for providing a large range of motion of the surgical instruments inserted therethrough.

In a preferred embodiment, the seal anchor member defines at least four longitudinal ports extending therethrough for accommodating surgical instruments.

### DESCRIPTION OF THE DRAWINGS

The above and other aspects, features, and advantages of the present disclosure will become more apparent in light of the following detailed description when taken in conjunction with the accompanying drawings in which:

FIG. 1 is a front perspective view of a surgical apparatus in accordance with the principles of the present disclosure illustrating a seal anchor member positioned relative to the tissue;

FIG. 2a is a front perspective view of the seal anchor member of FIG. 1 in the maximum folded state;

FIG. 2b is a front perspective view of the seal anchor member of FIG. 1 in an intermediate folded state between the maximum and minimum folded states;

FIG. 2c is a front perspective view of the seal anchor member of FIG. 1 in the minimum folded state;

FIG. 3 is a front perspective view of the seal anchor member of FIG. 1 illustrating the trailing end of the seal anchor member;

FIG. 4 is a perspective view of the seal anchor member of FIG. 1 illustrating the slot and the aperture of the trailing end;

FIG. 5 is a top perspective view of the seal anchor member of FIG. 1 illustrating a plurality of ports extending longitudinally therethrough;

FIG. 6 is a side cross-sectional view of the seal anchor member of FIG. 1 illustrating a port that extends longitudinally through the leading end and the intermediate portion of the seal anchor member;

FIG. 7 is a front prospective view of an alternate embodiment of the seal anchor member shown in an unfolded state;

FIG. 8 is a top prospective view of the seal anchor member of FIG. 7;

FIG. 9 is a front perspective view of the seal anchor member of FIG. 7 shown in an unfolded state and shown disposed within a tissue tract having first thickness;

FIG. 10 is a partially cutaway front perspective view of the seal anchor member of FIG. 7 shown in an unfolded state and shown disposed within a tissue tract having a second thickness;

FIG. 11 is a partially cutaway front perspective view of the seal anchor member of FIG. 7 shown in a folded state and shown disposed within the tissue tract of FIG. 10;

FIG. 12 is a perspective view of the seal anchor member of FIG. 7 shown in an unfolded state and shown disposed within the tissue tract of FIG. 10;

FIG. 13 is a perspective view of the seal anchor member of FIG. 7 illustrated in a folded state and shown disposed within the tissue tract of FIG. 10;

FIG. 14 is a front prospective view yet another embodiment of a seal anchor member shown positioned relative to the tissue;

FIG. 15 is a top perspective view of the seal anchor member of FIG. 14;

FIG. 16 is a front prospective view of an a still further embodiment of the seal anchor member; and

FIG. 17 is a top perspective view of the seal anchor member of FIG. 16.

### DETAILED DESCRIPTION

Particular embodiments of the present disclosure will be described herein with reference to the accompanying drawings. As shown in the drawings and as described throughout the following description, and as is traditional when referring to relative positioning on an object, the term “proximal” or “trailing” refers to the end of the apparatus that is closer to the user and the term “distal” or “leading” refers to the end of the apparatus that is farther from the user. In the following description, well-known functions or constructions are not described in detail to avoid obscuring the present disclosure in unnecessary detail.

One type of minimal invasive surgery described herein employs a device that facilitates multiple instrument access through a single incision. This is a minimally invasive surgical procedure, which permits a user to operate through a single entry point, typically the patient's navel. The disclosed procedure involves insufflating the body cavity and positioning a portal member within, e.g., the navel of the patient. Instruments including an endoscope and additional instruments such as graspers, staplers, forceps or the like may be introduced within the portal member to carry out the surgical procedure. An example of such a surgical portal is disclosed in commonly assigned U.S. patent application Ser. No. 12/244,024, Pub. No. US 2009/0093752 A1, filed Oct. 2, 2008, the entire content of which is hereby incorporated by reference herein.

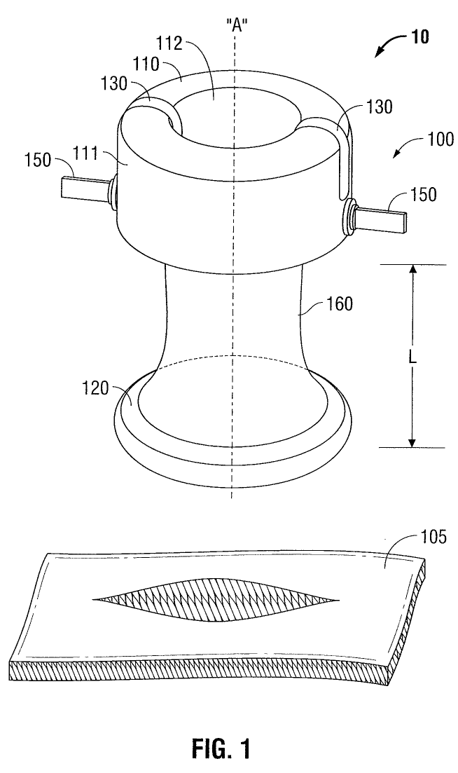

Referring now to the drawings, in which like reference numerals identify identical or substantially similar parts throughout the several views, FIG. 1 illustrates a surgical apparatus 10 comprising a seal anchor member 100 in accordance with the principles of the present disclosure. Seal anchor member 100 is adapted for insertion within a tissue tract 105, e.g., through the abdominal or peritoneal lining in connection with a laparoscopic surgical procedure. Seal anchor member 100 will be described in greater detail hereinbelow

With reference to FIG. 1, seal anchor member 100 defines a longitudinal axis “A” and a length “L”. The length “L” describes the distance of the portion of the seal anchor member 100 that can be inserted through the tissue tract 105. Seal anchor member 100 has respective trailing and leading ends 110, 120 and an intermediate portion 160 disposed between the trailing and leading ends 110, 120. Seal anchor member 100 may be made from a semi-resilient, disposable, compressible, and flexible type material, for example, but not limited to, a suitable foam, gel material, or soft rubber having sufficient compliance to form a seal about one or more surgical objects, and also establish a sealing relation with tissue and with the surgical object. In one embodiment, the foam includes a polyisoprene material. As shown in FIG. 1, seal anchor member 100 may define a substantially hourglass shape. However, it is contemplated that the seal anchor member 100 may define other configurations both prior and subsequent to insertion within tissue.

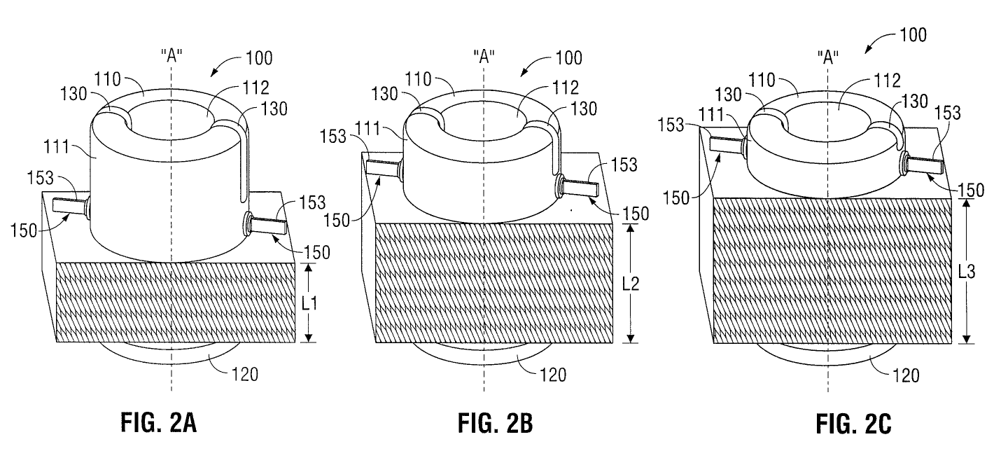

Due to the flexible and semi-resilient characteristics of seal anchor member 100, the length “L” of seal anchor member 100 can be adjusted to be suitable for tissues of different thicknesses. In one embodiment, as illustrated in FIG. 1, to configure seal anchor member 100 to have different lengths, trailing end 110 is configured to fold at any position along the longitudinal axis thus resulting in various seal anchor member lengths. As best illustrated in FIGS. 2a-2c, trailing end 110 is configured to define a plurality of folded states. Each of the three folded states in FIGS. 2a-2c corresponds to a different length of seal anchor member 100: “L1”, “L2” and “L3”. Trailing end 110 in each folded state defines an outer surface 111 and an inner surface 112. The outer surface 111 is formed by the portion of the trailing end 110 that is folded along the longitudinal axis “A”, whereas the inner surface 112 is formed by the portion of the trailing end 110 that is not yet folded. As the trailing end 110 transits among different folded states, portions of the outer surface 111 may gradually merge into the inner surface 112, and vice versa.

The plurality of the folded states range between a maximum folded state as shown in FIG. 2a and a minimum folded state as shown in FIG. 2c. The maximum folded state describes a state in which a maximum portion of the trailing end 110 is folded. Accordingly, the length “L” of seal anchor member 100 is minimized, thus resulting in a minimum length “L” of seal anchor member 100. In contrast, the minimum folded state happens when a minimum portion of the trailing end 110 is folded, resulting in a maximum length “L” of seal anchor member 100. Thus, seal anchor member 100 can be configured to different lengths, and it can be adapted to different tissue thicknesses.

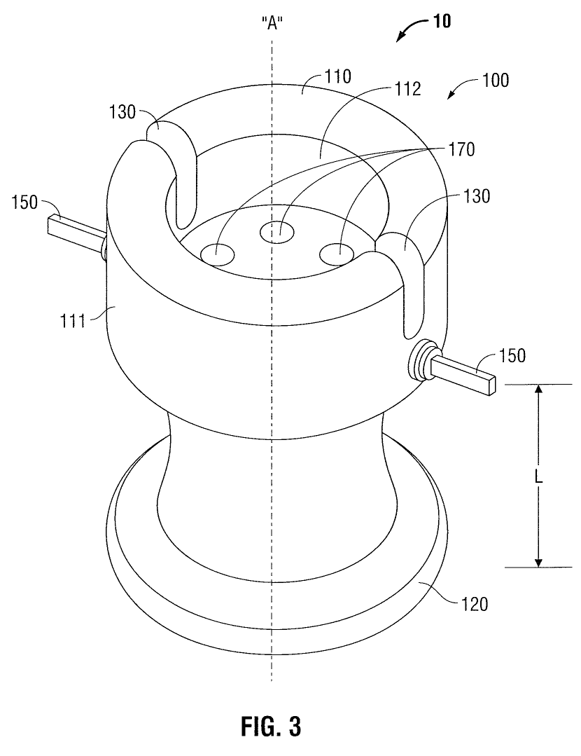

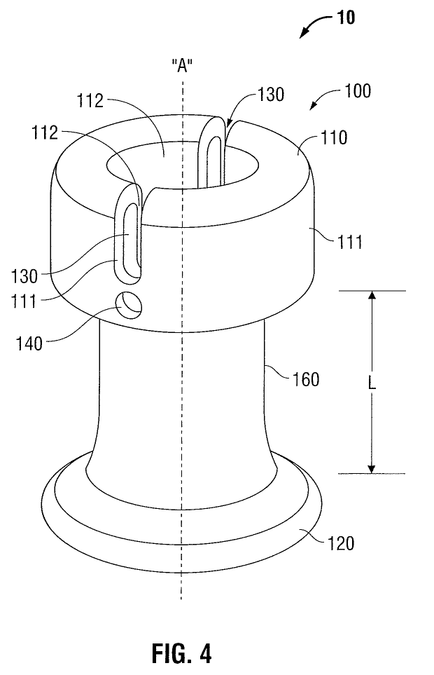

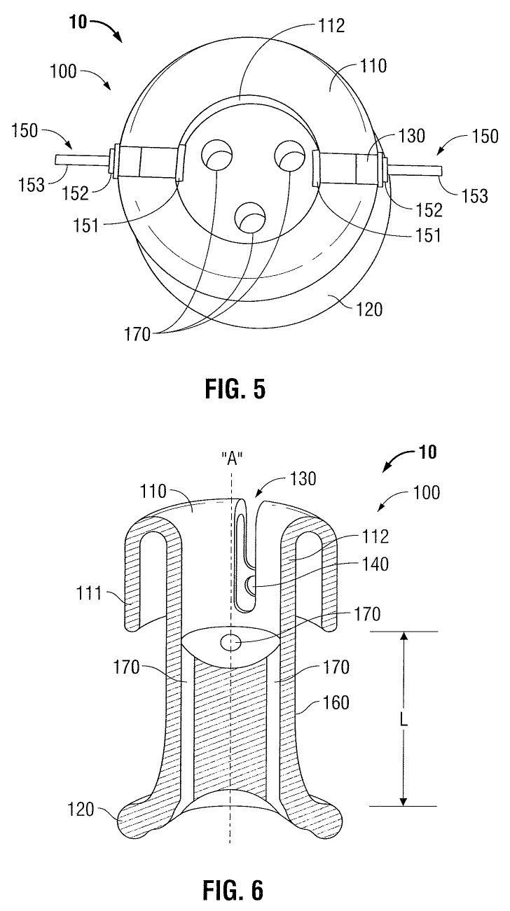

To facilitate transition between the maximum and minimum folded state, the trailing end 110 defines two slots 130, as illustrated in FIG. 3. Each slot 130 spans across the outer surface 111 and the inner surface 112 of the trailing end 110. The two slots 130 are diametrically opposed on the trailing end 110. As illustrated in FIG. 4, each slot 130 corresponds to an aperture 140 which is defined by the trailing end 110. Each aperture 140 is positioned directly beneath its corresponding slot 130 on the outer surface 111 of the trailing end 110. With reference to FIG. 5, each slot 130 further corresponds to a pin 150 which is disposed through the outer surface 111 and the inner surface 112 of the trailing end 110. Each pin 150 includes a head 151, a body 152, and a handle 153. The pin 150 is disposed through the outer surface 111 by having its body 152 positioned through the aperture 140 which is defined beneath the pin's 150 corresponding slot 130. The body 152 is attached firmly to the outer surface 111 of the trailing end 110. Further, the pin 150 protrudes beyond the outer surface and forms the handle 153 which can be used by a surgeon to adjust the position of pin 150 along the longitudinal axis “A.” Further, the pin 150 extends through its corresponding slot 130 defined on the inner surface 112. The portion of the pin 150 that extends beyond the inner surface 112 forms the head 151 of pin 150. The head 151 has a diameter greater than that of the body 152. The head 151 is placed against the inner surface 112 of the trailing end 110.

With continued reference to FIG. 5, the pin 150 is configured to slide through the slot 130 by lifting the handle 153 up and down along the longitudinal axis “A.” Essentially, the slot 130 provides a passageway that guides the pin 150 to slide longitudinally under the control of the handle 153. As the pin 150 slides from one end of the slot 130 to the other end of the slot 130, the trailing end 110 undergoes a transition between the maximum folded state and the minimum folded state. Accordingly, the seal anchor member 100 transits between the minimum length “L” and the maximum length “L”. Further details regarding this transition are explained below.

With reference to FIGS. 2a-2c, as the handle 153 is lifted in an upward direction along the longitudinal axis “A”, portions of the outer surface 111 gradually merge into the inner surface 112, thus, transiting the trailing end 111 from a more folded state, as shown in FIG. 2a, to a less folded state, as shown in FIG. 2c, thereby increasing the length “L” of the seal anchor member 100. In contrast, as the handle 153 is pushed in a downward direction along the longitudinal axis “A”, portions of the inner surface 112 gradually merge into the outer surface 111, thus, transiting the trailing end from a less folded state, shown in FIG. 2c, to a more folded state, as shown in FIG. 2a, thereby decreasing the length “L” of the seal anchor member 100. Thus, the user can adjust the length “L” of the seal anchor member 100 by adjusting the handle 153 along the longitudinal axis “A” until a desired length is reached.

With reference to FIGS. 5 and 6, the trailing end 110 exhibits an elongated, tubular structure defining a hollow center region, as shown in FIG. 6. Seal anchor member 100 further includes at least on longitudinal port 170 extending along the longitudinal axis “A” of seal anchor member 100 between the leading end 120 and the intermediate portion 160. The ports 170 are configured symmetrically with respect to the longitudinal axis “A”. The ports 170 are spaced equidistant from the longitudinal axis “A”. Each port 170 may be spaced equidistant from its neighboring ports. The longitudinal ports 170 are dimensioned to receive a surgical object, e.g. a surgical instrument (not shown) therethrough. Upon introduction of an instrument or surgical object (not shown) through a respective port 170, the inner surface portions defining the port 170 establish and maintain a substantial sealed relation about the instrument or surgical object.

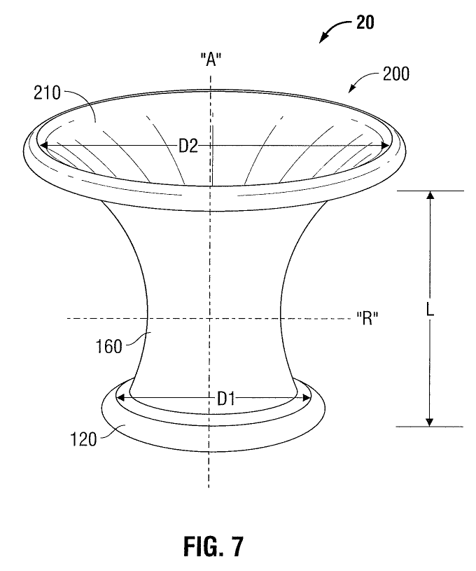

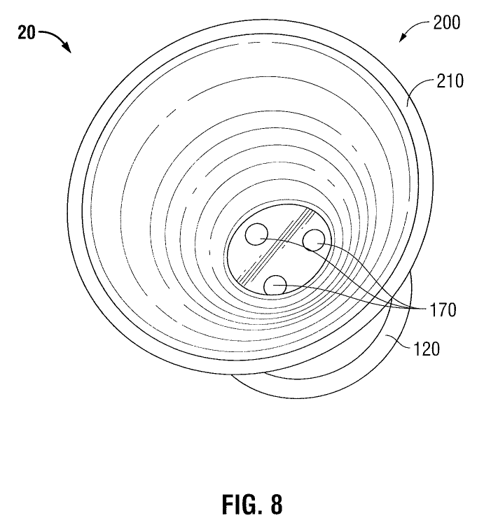

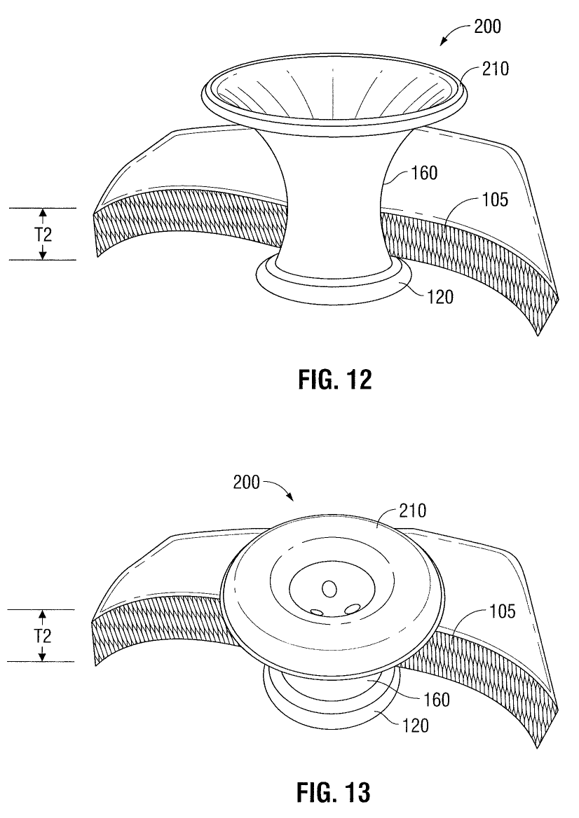

Turning now to FIGS. 7-13, a surgical apparatus 20 including a seal anchor member 200 will now be described. Seal anchor member 200 includes a leading end 120, an intermediate portion 160, and a trailing end 210. A plurality of ports 170 is disposed between the intermediate portion 160 and the leading end 120 as illustrated in FIG. 8. As illustrated in FIG. 7, the trailing end 210 has an unfolded state in which the trailing end 210 is fully unfurled. The seal anchor member 200 reaches its maximum length “L” when the trailing end 210 is in the unfolded state. The leading end 120 defines a radial diameter “D1” relatively smaller than a radial diameter “D2” defined by the trailing end 210. This particular configuration of the seal anchor member 200 with one end substantially small and the other end substantially large provides many benefits to surgical procedures. The leading end 120 with its relatively small dimension provides easy insertion and removal of the seal anchor member 200 through skin tissues, thus, reducing the time required to place and/or displace the seal anchor member 200 through the incisions during surgical operations and reducing tissue trauma. The trailing end 210 with its relatively large dimension creates a wide open space above the plurality of ports 170. As a result, the trailing end 210 provides the surgeon a large free space to manipulate portions of the surgical instruments positioned above the ports 170, ultimately resulting in a significantly increased range of motion of the surgical instruments inserted through the seal anchor member 200 and facilitating off-axis motions of the surgical instruments. The relatively large trailing end 210 also facilitates transition among the plurality of the folded states as well as the unfolded state.

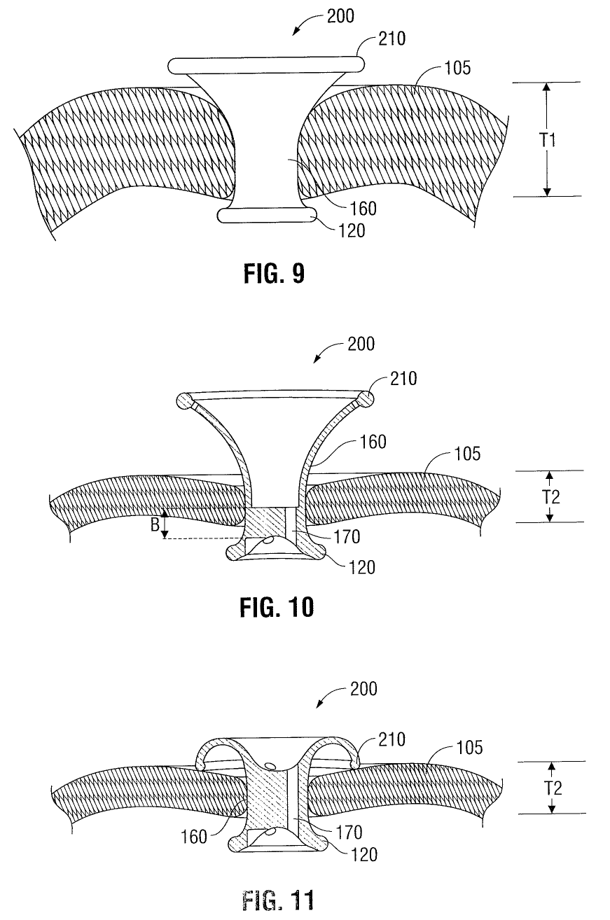

As shown in FIGS. 9-13, adjustment of the length of the seal anchor member 200 to accommodate tissue tracts 105 having different thicknesses. As seen in FIG. 9, tissue tract 105 has a thickness T1 that corresponds to the overall length of seal anchor member 200 in an unfolded state. In such a situation, where the thickness of the tissue tract 105 corresponds to the unfolded length of the seal anchor member 200, the seal anchor member 200 need not be folded. However, in situations in which the tissue tract 105 has a thickness, e.g., thickness T2 (FIGS. 10-13), that is less than the unfolded length of the seal anchor member 200, adjustment of the overall length of the seal anchor member 200 may be made to approximate thickness T2, as is illustrated in FIGS. 11 and 13. As illustrated in FIGS. 11 and 13, the trailing end 210 is folded such that the trailing end 210 is moved towards the surface of the tissue tract 105, thereby reducing the distance between trailing and leading ends 210, 120 and substantially approximating thickness T2.

In a preferred embodiment, the surgical apparatus exhibits a coring configuration as illustrated in FIG. 10, wherein the trailing end 210 and the intermediate portion 160 define a large free, open space in the center region of the seal anchor member 200 as if a large amount of materials has been removed therefrom. The intermediate portion 160 is filled with only a small amount of materials within the region “B” adjacent to the leading end 120, as indicated in FIG. 10, for defining the port 170. Therefore, the port 170 in this particular embodiment is relatively small along the longitudinal axis “A.” The coring configuration increases the flexibility of the seal anchor member 200 such that the overall shape of the seal anchor member 200 can be easily manipulated. For instance, due to the large empty space within the seal anchor member 200, the seal anchor member 200 can be easily squeezed to facilitate its insertion through incisions. Since the seal anchor member 200 can be easily reduced to a small dimension due to squeezing, the incision through which the seal anchor member 200 to be placed can be created to have a relatively small opening thereby reducing the trauma experienced by the patients and subsequently reducing the patients' recovery time required to heal the incision. Further, the large free space defined above the port 170 increases the maneuverability and the range of motion of the surgical instruments inserted through the seal anchor member 200 and also significantly facilitates off-axis motions of the surgical instruments.

In a certain embodiment, the seal anchor member defines a substantially large length, such that the intermediate portion of the surgical apparatus is substantially lengthy along the longitudinal axis “A” of the seal anchor member between the trailing and leading ends. The seal anchor member further includes at least one longitudinal port substantially lengthy along the longitudinal axis “A” thereof between the intermediate portion and the leading end or between the trailing end and the leading end. The surgical apparatus of this embodiment is particularly designed to accommodate unusually thick tissues for purposes to be used in bariatric related treatment procedures. Obese patients have significantly thick tissues compared to patients of normal weight. During bariatric related treatment procedures, an incision is initially created off the midline for providing an access to the patient's body cavity. Access devices taught by the prior art are oftentimes not tall enough to be placed across the entire abdominal walls of obese patients. Thus, the prior access device cannot be securely placed within incisions, thereby adversely influencing the operation of bariatric procedures. The seal anchor member with a substantial length solves this problem. The seal anchor member can be securely placed at the incision extending across a very thick abdominal wall for introducing surgical instruments therethrough to manipulate tissues or organs within the body cavity. Thus, the seal anchor member is securely fit with respect to the incision, resulting in a stable state facilitating introduction of surgical instruments therethrough for performing bariatric procedures.

In a preferred embodiment, the seal anchor member defines at least four ports 170, with at least one port for accommodating an instrument connected to an insufflation or evacuation source.

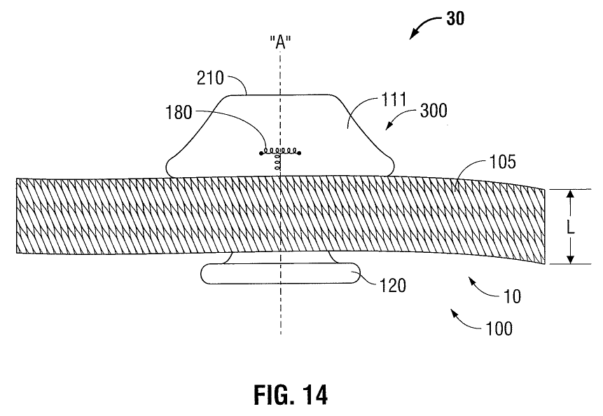

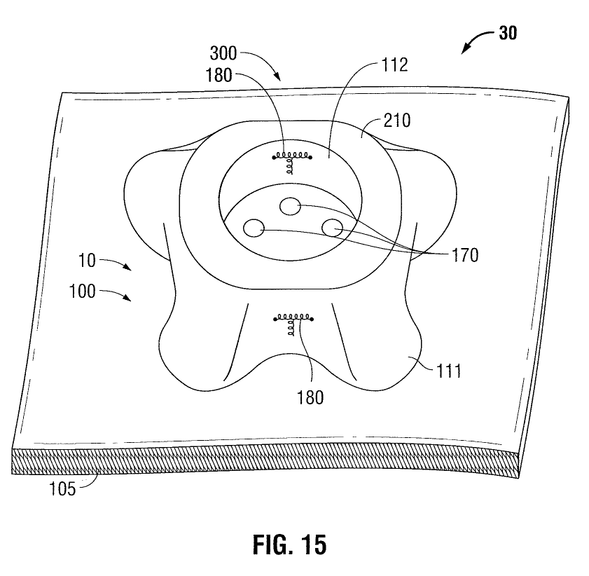

In a further embodiment, as illustrated in FIGS. 14 and 15, a surgical apparatus 30 includes a seal anchor member 300 including a leading end 120, a trailing end 210, an inner surface 112, an outer surface 111, and a suture 180 adapted to maintain a selected folded state. Upon folding the trailing end 210 to achieve a desired folded state, suture 180 may be used to connect the inner surface 112 and the outer surface 111 of the trailing end 110 together for purposes of maintaining the selected folded state.

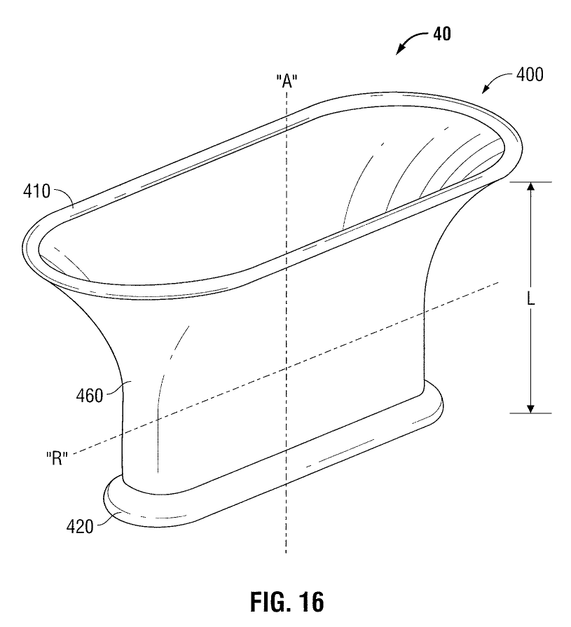

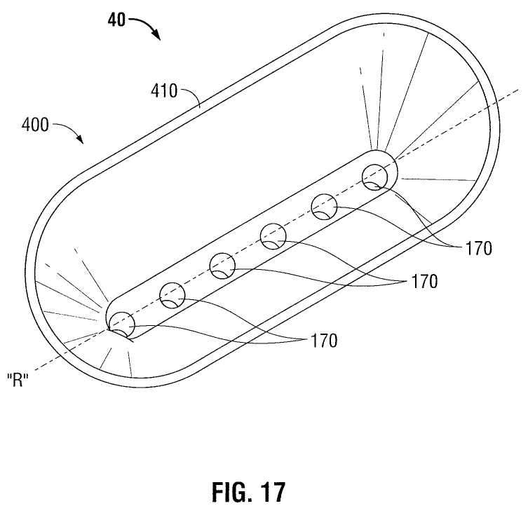

In a still further embodiment, as illustrated in FIGS. 16 and 17, a surgical apparatus 40 includes a seal anchor member 400. Seal anchor member 400 exhibits an elongated hourglass configuration and is substantially symmetrical with respect to radial axis “R.” A plurality of ports 170 is disposed within the seal anchor member 400 and are arranged in a linear fashion along radial axis “R,” each port 170 including a lumen that is substantially parallel to longitudinal axis “A” of the seal anchor member 400. The ports 170 may be equidistantly spaced apart.

Different embodiments of the disclosure may be combined with one another based on the particular needs of the patients to achieve optimal results of the surgical procedures. In one example, in bariatric related procedures, the seal anchor member may define a coring configuration having a substantial length for accommodating thick abdominal walls, and may comprise four longitudinal ports. In another example associated with bariatric related procedures, the seal anchor member may define a coring configuration having a substantial length for accommodating thick abdominal walls, and may further comprise a relatively small leading end, a relatively large trailing end and four longitudinal ports extending therethrough. Any of the presently disclosed embodiments may be used in procedures where access is achieved through a naturally occurring orifice (e.g. vagina or anus).

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical apparatus for positioning within a tissue tract accessing an underlying body cavity, which comprises:
a seal anchor member defining a longitudinal axis and a length, the seal anchor member including a first end and a second end, the first end configured to fold along the longitudinal axis, the first end defining a plurality of states, each state corresponding to a different length of the seal anchor member.

2. The surgical apparatus according to claim 1 wherein the plurality of states comprise a plurality of folded states.

3. The surgical apparatus according to claim 2 wherein the plurality of folded states include a maximum folded state such that the maximum folded state corresponds to a minimum length of the seal anchor member.

4. The surgical apparatus according to claim 2 wherein the plurality of folded states include a minimum folded state such that the minimum folded state corresponds to a maximum length of the seal anchor member.

5. The surgical apparatus according to claim 2 wherein each folded state has an outer surface and an inner surface of the first end.

6. The surgical apparatus according to claim 5 wherein each folded state is maintained by connecting the outer surface and the inner surface of the first end.

7. The surgical apparatus according to claim 2 wherein the first end defines a slot configured to facilitate transition within the plurality of folded states.

8. The surgical apparatus according to claim 2 wherein the first end defines an aperture through which a pin is configured to facilitate transition within the plurality of folded states.

9. The surgical apparatus according to claim 1 wherein the plurality of states comprise an unfolded state, wherein the unfolded state corresponds to a maximum length of the seal anchor member.

10. The surgical apparatus according to claim 1 further comprising an intermediate portion disposed between the first and second ends along the longitudinal axis.

11. The surgical apparatus according to claim 10 further comprising at least one longitudinal port extending between the intermediate portion and the second end and being configured for substantially sealed reception of an object therein.

12. The surgical apparatus according to claim 11 wherein the at least one longitudinal port is configured symmetrically with respect to the longitudinal axis.

13. The surgical apparatus according to claim 11 wherein the at least one longitudinal port is arranged in a linear fashion along a radial axis of the seal anchor member.

14. The surgical apparatus according to claim 1 wherein the trailing end exhibits a tubular configuration.

15. The surgical apparatus according to claim 1 wherein the seal anchor member exhibits an hourglass configuration.

16. The surgical apparatus according to claim 1 wherein the seal anchor member exhibits an hourglass configuration elongated in a radial axis of the seal anchor member.
